(19) United States
(12) Patent Application Publication
(10) Pub. No.: US 2024/0342105 A1
(43) Pub. Date: Oct. 17, 2024

(54) IONIZABLE LIPID BASED ON CYCLOHEXANEDIAMINE AND LIPID NANOPARTICLE, AND PREPARATION METHOD THEREFOR AND USE THEREOF

(71) Applicant: SHANDONG UNIVERSITY, Jinan (CN)

(72) Inventors: Xinyi JIANG, Jinan (CN); Ying LIU, Jinan (CN); Chunwei TANG, Jinan (CN); Longyu BO, Jinan (CN); Kun ZHAO, Jinan (CN); Chen CHEN, Jinan (CN); Weiqiang JING, Jinan (CN); Ying ZHI, Jinan (CN); Xiaotian ZHAO, Jinan (CN); Maosen HAN, Jinan (CN)

(73) Assignee: SHANDONG UNIVERSITY, Jinan (CN)

(21) Appl. No.: 18/630,721

(22) Filed: Apr. 9, 2024

(30) Foreign Application Priority Data
Apr. 12, 2023 (CN) 2023103859228

(51) Int. Cl. A61K 9/51; A61K 31/7105; C07C 237/22; B82Y 40/00

(52) U.S. Cl. CPC A61K 9/5123; A61K 9/5192; A61K 31/7105; B82Y 40/00; C07C 237/22; B82Y 5/00

(57) ABSTRACT

An ionizable lipid based on cyclohexanediamine and a lipid nanoparticle, and a preparation method therefor and use thereof. The ionizable lipid based on cyclohexanediamine is a compound represented by formula (a),

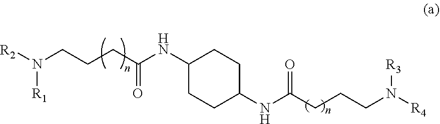

wherein n is independently selected from natural numbers of 0-8; R₁, R₂, R₃ and R₄ are independently selected from C₈-C₂₄ alkyl, C₈-C₂₄ alkyl substituted by a substituent group, C₈-C₂₄ alkenyl, C₈-C₂₄ alkenyl substituted by a substituent group, and C₈-C₂₄ alkynyl and C₈-C₂₄ alkynyl substituted by a substituent group. The lipid nanoparticle formed by the ionizable lipid based on cyclohexanediamine has the advantages of biodegradability, high in-vivo and in-vitro transfection efficiency, and the like, and has a good clinical application prospect.

### TECHNICAL FIELD

The present invention belongs to the technical field of biological medicines, relates to the drug delivery technology, and particularly relates to an ionizable lipid based on cyclohexanediamine and a lipid nanoparticle, and a preparation method therefor and use thereof.

### BACKGROUND

Information for disclosing this background art section is only for the purpose of increasing understanding of the general background of the present invention, and is not necessarily regarded as an acknowledgement or any form of suggestion that the information constitutes the prior art already known to those of ordinary skill in the art.

A lipid nanoparticle (LNP) delivery technology can realize the efficient delivery of a nucleic acid drug. LNP typically consists of four components of an ionizable lipid, a helper phospholipid, cholesterol, and a PEG lipid. Among them, the ionizable lipid material is a key component, which is useful for protecting nucleic acids and facilitating their transport in vivo. A gene therapy has the problems that the nucleic acid drug is easily degraded by a nuclease in blood plasma and tissues, causes immunogenicity, and is difficult to enter cells through membranes. Therefore, it is of important significance to develop an efficient and safe nucleic acid drug delivery system for the treatment of gene diseases and protein overexpression/deletion diseases without specific drugs, including preventive diseases, hereditary diseases, tumors and other diseases.

### SUMMARY

In order to solve the shortcomings of the prior art, the present invention aims to provide an ionizable lipid based on cyclohexanediamine and a lipid nanoparticle, and a preparation method therefor and use thereof. The lipid nanoparticle formed by the ionizable lipid based on cyclohexanediamine provided by the present invention has the advantages of biodegradability, high in-vivo and in-vitro transfection efficiency, and the like, and has a good clinical application prospect.

To achieve the above objective, the technical solution of the present invention is:
On a first aspect, an ionizable lipid based on cyclohexanediamine, being a compound represented by formula (a), a pharmaceutically acceptable salt of the compound represented by formula (a), a stereoisomer of the compound represented by formula (a), a tautomer of the compound represented by formula (a), a solvate of the compound represented by formula (a), a chelate of the compound represented by formula (a), a non-covalent complex of the compound represented by formula (a), or a prodrug of the compound represented by formula (a),

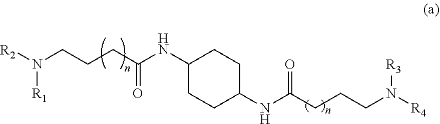

wherein n is independently selected from natural numbers of 0-8; and
R₁, R₂, R₃ and R₄ are independently selected from C₈-C₂₄ alkyl, C₈-C₂₄ alkyl substituted by a substituent group, C₈-C₂₄ alkenyl, C₈-C₂₄ alkenyl substituted by a substituent group, and C₈-C₂₄ alkynyl and C₈-C₂₄ alkynyl substituted by a substituent group.

On the other aspect, a method for preparing the ionizable lipid based on cyclohexanediamine, comprising the steps of obtaining the compound represented by formula (a) according to the following reaction formula,

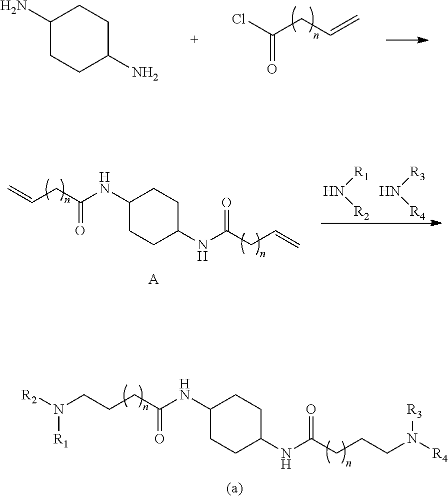

wherein n is independently selected from natural numbers of 0-8; and
R₁, R₂, R₃ and R₄ are independently selected from C₈-C₂₄ alkyl, C₈-C₂₄ alkyl substituted by a substituent group, C₈-C₂₄ alkenyl, C₈-C₂₄ alkenyl substituted by a substituent group, and C₈-C₂₄ alkynyl and C₈-C₂₄ alkynyl substituted by a substituent group.

On a third aspect, a lipid nanoparticle, comprising the ionizable lipid based on cyclohexanediamine, a helper lipid, a sterol, and a PEG lipid.

On a fourth aspect, a pharmaceutical composition, comprising the ionizable lipid based on cyclohexanediamine or the lipid nanoparticle, and an active ingredient, wherein the active ingredient is a nucleic acid drug.

On a fifth aspect, use of the lipid nanoparticle or the pharmaceutical composition in the preparation of a drug.

The beneficial effects of the present invention are:

1. The present invention improves the structure of the ionizable lipid molecule by introducing cyclohexanediamine and a stereisomer thereof to obtain a novel ionizable lipid compound. The six-membered ring structure of the compound is favorable for the nucleic acid drug to smoothly enter cells. Amide bonds in the structure can be quickly hydrolyzed by enzymes in vivo, are easy to be cleaned by metabolism, and have excellent biodegradability, biocompatibility and degradability. The compound can obtain hydrogen protons under the acidic condition, has electropositivity, can be combined with the electronegative nucleic acid molecule through electrostatic interaction, increases the stability of the nucleic acid drug, prolongs the circulation time thereof in vivo, improves the pharmacokinetic characteristics, has no obvious toxic or side effects, and has a good clinical application prospect.

2. The ionizable lipid provided by the present invention is prepared through a simple Michael addition synthesis and a condensation reaction with different branched-chain lengths. The raw material cost is low, the synthesis steps are simple, and the product is convenient to separate and easy to store.

3. The LNP prepared from the ionizable lipid, the helper phospholipid, the cholesterol and the PEG lipid provided by the present invention has a more excellent nucleic acid carrier performance, and can effectively deliver nucleic acid drugs such as siRNA, mRNA, pDNA and the like into cells to play a role.

4. The preparation method for the lipid nanoparticle is convenient and rapid, has lower equipment requirement, and is reliable in process.

### BRIEF DESCRIPTION OF THE DRAWINGS

As a part of the present invention, the accompanying drawings of the description provide further understanding of the present invention. The schematic examples of the present invention and description thereof are intended to explain the present invention and are not intended to constitute an improper limitation to the present invention.

FIG. 1 shows characterization of particle sizes of mRNA-LNPs prepared from different ionizable lipids in an example of the invention;

FIG. 2 shows characterization of potentials of mRNA-LNPs prepared from different ionizable lipids in an example of the invention;

FIG. 3 shows characterization of encapsulation efficiencies of mRNA-LNPs prepared from different ionizable lipids in an example of the invention; and

FIG. 4 shows characterization of transfection efficiencies of mRNA-LNPs prepared from different ionizable lipids in an example of the invention.

### DETAILED DESCRIPTION

It should be pointed out that the following detailed description is illustrative and is intended to provide further explanation of the present invention. Unless otherwise defined, all technical and scientific terms used herein have the same meanings as commonly understood by those of ordinary skill in the art to which the present invention belongs.

It should be noted that the terms used herein are merely used for describing the specific implementations, but are not intended to limit exemplary implementations of the present invention. As used herein, a singular form is intended to include a plural form unless otherwise indicated obviously in the context. Furthermore, it should be further understood that the terms “includes” and/or “including” used in this specification specify the presence of features, steps, operations, devices, components and/or a combination thereof.

The term “nucleic acid” as used herein relates to polymers of deoxyribonucleotides (DNA), ribonucleotides (RNA), and modified forms thereof, in the form of an individual fragment or as a component of a larger construct, and in the form of a straight chain or a branched chain, a single chain, a double chain, a triple chain or a hybrid thereof. The term also includes an RNA/DNA hybrid.

The term “lipid” as used herein refers to a group of organic compounds, including but not limited to esters of fatty acids, and is generally characterized by being poorly soluble in water but soluble in many organic solvents.

The term “lipid nanoparticle” as used herein refers to a particle having at least one nanoscale size, which comprises at least one lipid.

The term “delivery system” as used herein refers to a formulation or composition that regulates the spatial, temporal and dose distribution of a biologically active ingredient in an organism.

The term “cyclohexanediamine” as used herein refers to a cycloalkane containing 6 carbon atoms in which 2 hydrogen atoms are substituted by amino groups.

The term “alkyl” as used herein refers to a saturated aliphatic hydrocarbon group including straight-chain and branched-chain alkyl groups. The substituent group of C8-24 alkyl groups is one or more halogen, hydroxyl, amino, alkoxycarbonyl, amido, alkylamido, dialkylamido, nitro, alkylamino, dialkylamino, carboxyl, thioalkyl, and a heteroatom substituent group (oxo and thioxo).

The term “alkenyl” as used herein refers to a saturated aliphatic hydrocarbon group including straight-chain and branched-chain alkenyl group. The substituent group of C8-24 alkenyl groups is one or more halogen, hydroxyl, amino, alkoxycarbonyl, amido, alkylamido, dialkylamido, nitro, alkylamino, dialkylamino, carboxyl, thioalkyl, and a heteroatom substituent group (oxo and thioxo).

The term “alkynyl” as used herein refers to not a saturated aliphatic hydrocarbon group including straight-chain and branched-chain alkynyl groups. The substituent group of C8-24 alkynyl groups is one or more halogen, hydroxyl, amino, alkoxycarbonyl, amido, alkylamido, dialkylamido, nitro, alkylamino, dialkylamino, carboxyl, thioalkyl, and a heteroatom substituent group (oxo and thioxo).

The term “substituted” as used herein refers to that one or more hydrogen atoms in a group are substituted independently of each other by a corresponding number of substituent groups.

The term “pharmaceutical adjuvant” as used herein is an excipient and an additive used in the manufacture of drugs and in the formulation of prescriptions, and a substance reasonably assessed in terms of safety and contained in a pharmaceutical formulation other than an active ingredient. The pharmaceutical adjuvant may be Arabic gum, syrup, lanolin, starch, magnesium chloride, cyclodextrin, decanedioic acid, dextrin, pharmaceutical calcium sulfate, glycerol, mannitol, sorbitol, inositol, mercaptan, tromethamine, phenol, m-cresol, benzyl alcohol, p-hydroxybenzoate ester, methyl p-hydroxybenzoate, tert-butanol, benzalkonium chloride, chlorobutanol, and thimerosal.

The term “stereoisomer” as used herein refers to an isomer resulting from different arrangement of atoms in a molecule in a spatial manner, and may be classified into cis-trans isomers and enantiomers, and can also be classified into enantiomers and diastereomers. The stereoisomer may be isomerism of double bonds of olefins, C═N double bonds, N═N double bonds, and cyclic compounds.

The term “tautomer” as used herein refers to a particular functional group isomer produced by the rapid movement of an atom in two positions in a molecule, usually in the form of a relatively stable isomer as its predominant form of existence. The tautomer may be enol-keto tautomerism, amide-imidic acid tautomerism, lactam-lactim tautomerism, amide-imidic acid tautomerism in a heterocyclic ring, enamine-iminenamine tautomerism, proton transfer tautomerism, annular tautomerism, ring-chain tautomerism, and valence tautomerism.

The term “chelate” as used herein refers to a complex having a cyclic structure obtained by chelation in which two or more ligands form a chelate ring with the same metal ion, and having stability similar to that of an aromatic ring. The chelate may be a complexone (including nitrilotriacetic acid (NTA), ethylenediaminetetraacetic acid (EDTA), etc.), dithizone, 8-hydroxyquinoline, 1,10-phenanthroline (C12H8N2), potassium sodium tartrate, ammonium citrate, polyphosphate, etc.

The term “prodrug” as used herein, also called a drug precursor, refers to a compound obtained by modifying the chemical structure of a drug, which has no or little activity in vitro and releases an active drug by enzymatic or non-enzymatic conversion in vivo to exert the efficacy. The prodrug can improve the bioavailability of the drug, increase the stability of the drug, reduce the toxic and side effect, promote the long-acting of the drug, and the like.

In order to develop highly efficient and safe nucleic acid drug delivery system, the present invention provides an ionizable lipid based on cyclohexanediamine and a lipid nanoparticle, and a preparation method therefor and use thereof.

A typical embodiment of the present invention provides an ionizable lipid based on cyclohexanediamine, being a compound represented by formula (a), a pharmaceutically acceptable salt of the compound represented by formula (a), a stereoisomer of the compound represented by formula (a), a tautomer of the compound represented by formula (a), a solvate of the compound represented by formula (a), a chelate of the compound represented by formula (a), a non-covalent complex of the compound represented by formula (a), or a prodrug of the compound represented by formula (a),

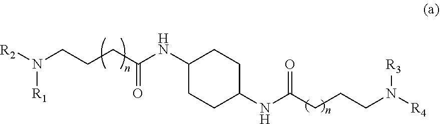

wherein n is independently selected from natural numbers of 0-8; and
R₁, R₂, R₃ and R₄ are independently selected from C₈-C₂₄ alkyl, C₈-C₂₄ alkyl substituted by a substituent group, C₈-C₂₄ alkenyl, C₈-C₂₄ alkenyl substituted by a substituent group, and C₈-C₂₄ alkynyl and C₈-C₂₄ alkynyl substituted by a substituent group.

The bond “ [symbol] ” in the chemical structure of formula (a) in the present invention represents an unspecified configuration, i.e., if a stereoisomer exists in the chemical structure, the bond “ [symbol] ” may be “ [symbol] ” or “ [symbol] ” or include the two configurations of “ [symbol] ” and “ [symbol] ” at the same time. In the chemical structure of the compound disclosed by the disclosure, the bond “ [symbol] ” does not specify a configuration, i.e., can be in a Z configuration or an E configuration, or include the two configurations at the same time.

In some examples, n is independently selected from 0 or 1.

In some examples, the following compounds are included:

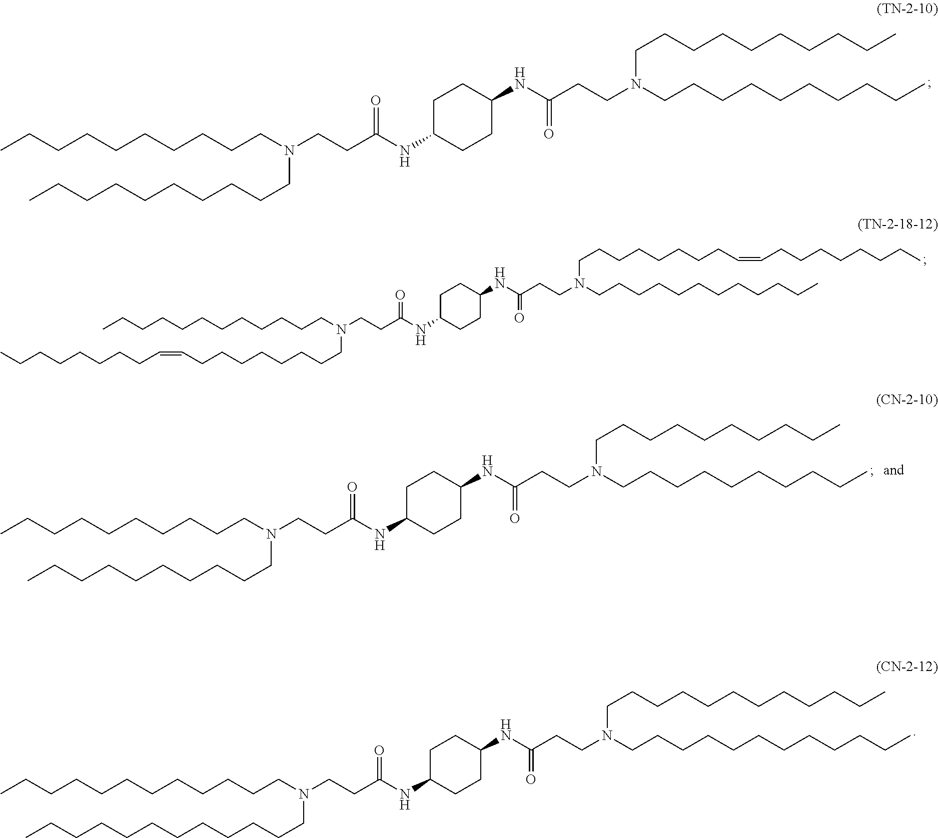

Another embodiment of the present invention provides a method for preparing the ionizable lipid based on cyclohexanediamine, comprising the steps of obtaining the compound represented by formula (a) according to the following reaction formula,

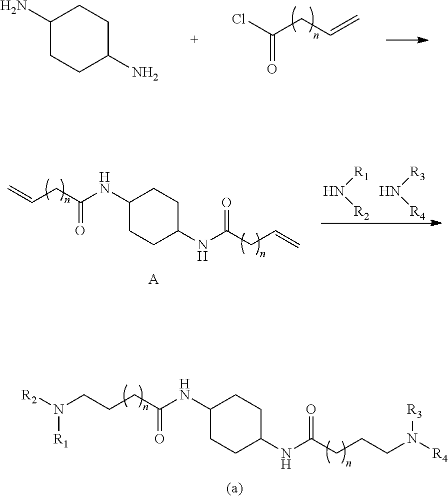

wherein n is independently selected from natural numbers of 0-8; and
R₁, R₂, R₃ and R₄ are independently selected from C₈-C₂₄ alkyl, C₈-C₂₄ alkyl substituted by a substituent group, C₈-C₂₄ alkenyl, C₈-C₂₄ alkenyl substituted by a substituent group, and C₈-C₂₄ alkynyl and C₈-C₂₄ alkynyl substituted by a substituent group.

In some examples, in the process of preparing an intermediate product A, materials are added under the ice bath condition and then reacted at room temperature.

In some examples, in the process of preparing the compound represented by formula (a) using the intermediate product A, the reaction temperature is 70-85° C., preferably, 75-80° C. The reaction time is 4-24 h, preferably 5-15 h.

In some examples, a solvent system of the reaction includes, but is not limited to, methanol, ethanol, isopropanol, benzene, toluene, xylene, pentane, hexane, octane, cyclohexanediamine, cyclohexanone, tolucyclohexanone, chlorobenzene, dichlorobenzene, dichloromethane, diethyl ether, propylene oxide, acetone, methyl butanone, methyl isobutyl ketone, N,N-dimethylformamide, N,N-dimethylacetamide, acetonitrile, pyridine, phenol, styrene, and triethanolamine. Preferably, the solvent of the reaction is dichloromethane, methanol, and N,N-dimethylformamide.

A third embodiment of the present invention provides a lipid nanoparticle, comprising the ionizable lipid based on cyclohexanediamine, a helper lipid, a sterol, and a PEG lipid.

In some examples, the helper lipid is distearoyl-sn-glycero-phosphoethanolamine, distearoyl phosphatidylcholine (DSPC), dioleoyl phosphatidylcholine (DOPC), dipalmitoyl phosphatidylcholine (DPPC), dioleoyl phosphatidylglycerol (DOPG), dipalmitoyl phosphatidylglycerol (DPPG), dioleoyl phosphatidylethanolamine (DOPE), palmitoyl oleoyl phosphatidylcholine (POPC), palmitoyl oleoyl phosphatidylethanolamine (POPE), dioleoyl phosphatidylethanolamine (DOPE), dioleoyl phosphatidylethanolamine 4-(N-maleimidomethyl)-cyclohexanediamine-1-carboxylate (DOPE-mal), dipalmitoyl phosphatidylethanolamine (DPPE), dimyristoyl phosphatidylethanolamine (DMPE), distearoyl-phosphatidylethanolamine (DSPE), monomethyl-phosphatidylethanolamine (e.g., 16-O-monomethyl PE), dimethyl-phosphatidylethanolamine (e.g., 16-O-dimethyl PE), 18-1-trans PE, 1-stearoyl-2-oleoyl-phosphatidylethanolamine (SOPE), hydrogenated soybean phosphatidylcholine (HSPC), egg phosphatidylcholine (EPC), dioleoyl phosphatidylserine (DOPS), sphingomyelin (SM), dimyristoyl phosphatidylcholine (DMPC), dimyristoyl phosphatidylglycerol (DMPG), distearoyl phosphatidylglycerol (DSPG), dierucoyl phosphatidylcholine (DEPC), palmitoyl oleoyl phosphatidylglycerol (POPG), di-trans oleoyl-phosphatidylethanolamine (DEPE), 1,2-dilauroyl-sn-glycerol-3-phosphoethanolamine (DLPE), 1,2-diphytanoyl-sn-glycero-3-phosphoethanolamine (DPHyPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebroside, ceryl phosphate, lysophosphatidylcholine, dilinoleoylphosphatidylcholine, or a mixture thereof. According to some embodiments, a non-cationic lipid is selected from one or any combination of dioleoyl phosphatidylcholine (DOPC), distearoyl phosphatidylcholine (DSPC), and dioleoyl-phosphatidylethanolamine (DOPE).

In some examples, the sterol includes, but is not limited to one or any combination of cholesterol, 20α-hydroxy cholesterol, and β-sitosterol. Preferably, the sterol is cholesterol and/or 20α-hydroxy cholesterol.

The PEG lipid is PEG and a modified lipid thereof. In some examples, the PEG lipid includes, but is not limited to: polyethylene glycol (PEG), 1-(monomethoxy-polyethylene glycol)-2,3-dimyristoyl glycerol (PEG-DMG or DMG-PEG), distearoylphosphatidylethanolamine-polyethylene glycol (DSPE-PEG), dipalmitoylphosphatidylethanolamine-methoxypolyethylene glycol (DPPE-PEG), dimethacrylate-polyethylene glycol (PEG-DMA), and 1,2-distearyloxypropyl-3-amine-N-[methoxy (polyethylene glycol)](PEG-DSA). Preferably, the PEG lipid is selected from DMG-PEG2000.

In some embodiments, the PEG lipid further includes a cationic lipid. The cationic lipid includes, or but is not limited to one any combination of trimethyl-2,3-dioleyloxypropylammonium chloride (DOTMA), bromide (DOTAP), methyl trimethyl-2,3-dioleyloxypropylammonium 4-(N,N-dimethylamino) butanoate (dilinoleyl)methyl ester (MC3), dimethyl-2,3-dioleyloxypropyl-2-(2-spermineformylamino)ethylammonium trifluoroacetate (DOSPA), trimethyldodecylammonium bromide (DTAB), trimethyltetradecylammonium bromide (TTAB), dimethyl-2-hydroxyethyl-2,3-bistetradecyloxypropylammonium bromide (DMRIE), N-(2-spermineformyll)-N′,N′-dioctadecylglycinamide (DOGS), 1,2-dioleyl-3-succinyl-sn-glycerocholine ester (DOSC), and 3β-[N—(N′,N′-dimethylaminoethyl)amidoformyl]cholesterol (DC-Chol).

In some examples, the mole percent of the ionizable lipid based on cyclohexanediamine is 0.1-100%, preferably 30-50%.

In some examples, the mole percent of the helper lipid is 0-99.9%, preferably 10-20%.

In some examples, the mole percent of the sterol is 0-99.9%, preferably 20-50%.

In some examples, the mole percent of the PEG lipid is 0-99.9%, preferably 2-5%.

A fourth embodiment of the present invention provides a pharmaceutical composition, comprising the ionizable lipid based on cyclohexanediamine or the lipid nanoparticle, and an active ingredient, wherein the active ingredient is a nucleic acid drug.

In some examples, the nucleic acid drug includes, but is not limited to, one or a combination of more of siRNA, mRNA, microRNA, circular mRNA, snRNA, snoRNA, tRNA, IRNA, gRNA, shRNA, piRNA, rasiRNA, hnRNA, long non-coding RNA, plasmid DNA, ceDNA, mini circle DNA, antisense oligonucleotides (ASOs), a DNA viral vector, a viral RNA vector, and a non-viral vector.

In some examples, the nucleic acid drug may also be a chemically modified polynucleotide. The chemical modification includes, but is not limited to chemical modification on a nucleic acid backbone, chemical modification on a base, and chemical modification on a ribose.

Specifically, the chemical modification on the nucleic acid backbone includes one or any combination of phosphorothioate diester linkage, morpholino ring substitution, dimethylamino phosphorodiester linkage, peptide nucleic acid, phosphorothioation, and the like.

Specifically, the chemical modification on the base includes one or any combination of m6A, N6, m5C, hm5C, Ψ, Nm, m3C, m7G, Cm, Gm, m5U, Um, and the like.

Specifically, the chemical modification on the ribose includes one or any combination of 2′-OMe, 2′-MOE, 2′-F, 2′-O-AP, 2′-O-m6Am, 2′-O-m3Um, PMO, and the like.

In some examples, the nucleic acid drug includes a polynucleotide comprising a steric structure. The polynucleotide comprising a steric structure includes, but is not limited to, one or any combination of locked nucleic acid (LNA), tricyclo DNA (tcDNA), glycerol nucleic acid (GNA), unlocked nucleic acid (UNA), threose nucleic acid (TNA), and the like.

In some examples, the chemical modification in the nucleic acid drug further includes, but is not limited to modification of any one or any combination of targeting groups: GalNac, mannose, galactose, RGD, PLGA, PEI, CPP, RVG, and the like.

A fifth embodiment of the present invention provide use of the lipid nanoparticle or the pharmaceutical composition in the preparation of a drug.

In some examples, the drug is used for treating a disorder including, but not limited to cancer, infection, an endocrine system disease, an autoimmune disease, a respiratory system disease, a neurodegenerative disease, inflammation, and a gene disease, preferably the genetic disease.

Specifically, the genetic disease includes, but is not limited to: sickle cell anemia, melanoma, hemophilia A (coagulation factor VIII (FVIII) deficiency) and hemophilia B (coagulation factor IX (FIX) deficiency), cystic fibrosis (CF), familial hypercholesterolemia (LDL receptor deficiency), hepatoblastoma, Wilson's disease, inherited hepatic metabolic diseases, LeschNyhan syndrome, thalassemia, pigmentary xeroderma, Fanconi anemia, pigmentary retinitis, ataxia telangiectasia, Bloom syndrome, retinoblastoma, mucopolysaccharidosis (e.g., Hurler syndrome (MPS-IH type), Scheie syndrome (MPS-IS type), Hurler-Scheie syndrome (MPS-IH/S type), Hunter syndrome (MPS-II type), Sanfilippo types A, B, C and D (MPS-IIIA, B, C and D), Morquio types A and B (MPS-IVA and MPS-IVB), Maroteaux-Lamy syndrome (MPS-VI type), Sly syndrome (MPS-VII type), hyaluronidase deficiency (MPS-IX type), Niemann-Pick disease types A/B, C1 and C2, Fabry disease, Schindler disease, GM2-gangliosidosis type II (Sandhoff disease), Tay-Sachs disease, metachromatic leukodystrophy, Krabbe disease, mucolipidosis types I, II/III and IV, sialic acid storage disease types I and II, glycogen storage disease types I and II (Pompe disease), Gaucher disease types I, II and III, Fabry disease, cystinosis, Batten disease, aspartylglucosaminuria, Salla disease, Danon disease (LAMP-2 deficiency), lysosomal acid lipase (LAL) deficiency, neuronal ceroid lipofuscinosis (CLN1-8, INCL and LINCL), sphingolipidosis, galactosialidosis, Parkinson's disease, Alzheimer's disease, Huntington's disease, spinocerebellar ataxia, spinal muscular atrophy, Freidrich's ataxia, Duchenne muscular dystrophy (DMD), Becker muscular dystrophy (BMD), dystrophic epidermolysis bullosa (DEB), ectonucleotide pyrophosphatase 1 deficiency, generalized arterial calcification of infancy (GACI), Leber congenital amaurosis, Stargardt macular dystrophy (ABCA4), ornithine transcarbamylase (OTC) deficiency, Usher syndrome, α-1 antitrypsin deficiency, progressive familial intrahepatic cholestasis (PFIC) type I (ATP8B1 deficiency), type II (ABCB11), type III (ABCB4) or type IV (TJP2), and cathepsin A deficiency.

In some examples, the mode of administration of the drug is systemic or local, including but not limited to any one or any combination of the following: oral administration, sublingual administration, rectal administration, vaginal administration, intravenous injection, intramuscular injection, subcutaneous injection, intraperitoneal injection, bone marrow injection, inhalation administration, intranasal administration, buccal administration, transdermal administration, mucosal administration, intraocular administration, otic administration, and the like.

A sixth embodiment of the present invention provides a method for delivering the pharmaceutical composition of the ionizable lipid or a salt thereof to a cell. The pharmaceutical composition is formulated in any form that specifically targets and/or transfects one or more target cells, tissues, and organs.

A mechanism that facilitates transfection of a target cell includes, for example, release of a membrane fusion group of a lipid bilayer membrane of a target cell and/or a proton sponge effect-mediated disruption.

In some examples, the target cell includes, but is not limited to one or more of the following cells: hepatocytes, hematopoietic cells, epithelial cells, endothelial cells, lung cells, bone cells, stem cells, mesenchymal cells, neural cells, heart cells, adipocytes, vascular smooth muscle cells, cardiac muscle cells, skeletal muscle cells, pituitary cells, synovial lining cells, ovarian cells, testicular cells, fibroblasts, B cells, T cells, reticulocytes, macrophages, neutrophils, eosinophils, basophils, and tumor cells. The target cell is a prokaryotic cell or a eukaryotic cell.

A seventh embodiment of the present invention provides a method for treating a disorder of a subject. The subject is administered with an effective amount of the pharmaceutical composition.

In some examples, the subject is an animal and/or a human.

In some examples, the disorder includes, but is not limited to cancer, infection, an endocrine system disease, an autoimmune disease, a respiratory system disease, a neurodegenerative disease, inflammation, and a gene disease, preferably the genetic disease.

Specifically, the genetic disease includes, but is not limited to: sickle cell anemia, melanoma, hemophilia A (coagulation factor VIII (FVIII) deficiency) and hemophilia B (coagulation factor IX (FIX) deficiency), cystic fibrosis (CF), familial hypercholesterolemia (LDL receptor deficiency), hepatoblastoma, Wilson's disease, inherited hepatic metabolic diseases, LeschNyhan syndrome, thalassemia, pigmentary xeroderma, Fanconi anemia, pigmentary retinitis, ataxia telangiectasia, Bloom syndrome, retinoblastoma, mucopolysaccharidosis (e.g., Hurler syndrome (MPS-IH type), Scheie syndrome (MPS-IS type), Hurler-Scheie syndrome (MPS-IH/S type), Hunter syndrome (MPS-II type), Sanfilippo types A, B, C and D (MPS-IIIA, B, C and D), Morquio types A and B (MPS-IVA and MPS-IVB), Maroteaux-Lamy syndrome (MPS-VI type), Sly syndrome (MPS-VII type), hyaluronidase deficiency (MPS-IX type), Niemann-Pick disease types A/B, C₁ and C₂, Fabry disease, Schindler disease, GM2-gangliosidosis type II (Sandhoff disease), Tay-Sachs disease, metachromatic leukodystrophy, Krabbe disease, mucolipidosis types I, II/III and IV, sialic acid storage disease types I and II, glycogen storage disease types I and II (Pompe disease), Gaucher disease types I, II and III, Fabry disease, cystinosis, Batten disease, aspartylglucosaminuria, Salla disease, Danon disease (LAMP-2 deficiency), lysosomal acid lipase (LAL) deficiency, neuronal ceroid lipofuscinosis (CLN1-8, INCL and LINCL), sphingolipidosis, galactosialidosis, Parkinson's disease, Alzheimer's disease, Huntington's disease, spinocerebellar ataxia, spinal muscular atrophy, Freidrich's ataxia, Duchenne muscular dystrophy (DMD), Becker muscular dystrophy (BMD), dystrophic epidermolysis bullosa (DEB), ectonucleotide pyrophosphatase 1 deficiency, generalized arterial calcification of infancy (GACI), Leber congenital amaurosis, Stargardt macular dystrophy (ABCA4), ornithine transcarbamylase (OTC) deficiency, Usher syndrome, α-1 antitrypsin deficiency, progressive familial intrahepatic cholestasis (PFIC) type I (ATP8B1 deficiency), type II (ABCB11), type III (ABCB4) or type IV (TJP2), and cathepsin A deficiency.

In some examples, the mode of administration of the drug is systemic or local, including but not limited to any one or any combination of the following: oral administration, sublingual administration, rectal administration, vaginal administration, intravenous injection, intramuscular injection, subcutaneous injection, intraperitoneal injection, bone marrow injection, inhalation administration, intranasal administration, buccal administration, transdermal administration, mucosal administration, intraocular administration, otic administration, and the like.

In order to make the technical solutions of the present invention more comprehensible to those skilled in the art, the technical solutions of the present invention are described in detail below with reference to specific examples.

Experimental methods in the following examples of the present invention which are not specified with specific conditions are generally carried out according to conventional conditions or conditions recommended by raw material or commodity manufacturers. Reagents not specified with specific origin are commercially available conventional reagents.

### Example 1: Preparation of TN Series of Ionizable Lipid Compounds

1) 5.0 mmol of trans-1,4-cyclohexanediamine, 11 mmol of triethylamine, and 25 mL of anhydrous dichloromethane were sequentially added into a 100-mL reaction bottle filled with magnetons and precooled under the ice bath condition. 11 mmol of acryloyl chloride was slowly dropwise added, the ice bath was removed after the dropwise adding of the acryloyl chloride was finished, and the reaction was performed at room temperature for 4 h. A solvent was removed using a rotary evaporator, a saturated sodium bicarbonate solution was added and stirred, and suction filtration was performed to obtain an intermediate product A with the yield of 95%.

2) 0.20 mmol of the intermediate product A, 0.44 mmol of an organic amine, and 0.50 mL of methanol were added to a 4-mL reaction flask containing magnetons, and the reaction was performed while stirring overnight at 75° C. The solvent was removed using the rotary evaporator and the product was separated by a thin layer chromatographic column (dichloromethane:methanol=10:1 by volume ratio) to obtain the following target products.

The intermediate product A was prepared using trans-1,4-cyclohexanediamine, the organic amine was didecylamine, the product was TN-2-10 (yield of 40%), and the structural characterization: 1H NMR (400 MHZ, CDCl3) δ 8.64 (s, 2H), 3.70 (s, 2H), 2.61 (t, J=5.5 Hz, 4H), 2.47-2.35 (m, 8H), 2.35-2.26 (m, 4H), 1.95 (d, J=6.2 Hz, 4H), 1.25 (s, 68H), 0.86 (t, J=6.7 Hz, 12H).

The intermediate product A was prepared using trans-1,4-cyclohexanediamine, the organic amine was N-9-octadecen-1-dodecylamine, the product was TN-2-18-12 (yield of 35%), and the structural characterization: 1H NMR (400 MHZ, CDCl3) δ 8.57 (s, 2H), 5.44-5.15 (m, 4H), 3.66 (s, 2H), 2.56 (t, J=5.9 Hz, 4H), 2.34 (t, J=7.6 Hz, 8H), 2.26 (t, J=5.8 Hz, 4H), 1.93 (dt, J=22.5, 7.1 Hz, 12H), 1.37 (s, 4H), 1.19 (s, 88H), 0.81 (t, J=6.6 Hz, 12H).

### Example 2: Preparation of CN Series of Ionizable Lipid Compounds

1) 5.0 mmol of cis-1,4-cyclohexanediamine, 11 mmol of triethylamine, and 25 mL of anhydrous dichloromethane were sequentially added into a 100-mL reaction bottle filled with magnetons and precooled under the ice bath condition. 11 mmol of acryloyl chloride was slowly dropwise added, the ice bath was removed after the dropwise adding of the acryloyl chloride was finished, and the reaction was performed at room temperature for 4 h. The reaction solution was extracted with a saturated sodium bicarbonate solution and ethyl acetate, and washed with saturated sodium chloride. The organic phase was dried over anhydrous sodium sulfate and separated by a thin layer chromatographic column (dichloromethane:methanol=10:1 by volume ratio) to obtain an intermediate product A with the yield of 37%.

2) 0.20 mmol of the intermediate product A, 0.44 mmol of an organic amine, and 0.50 mL of methanol were added to a 4-mL reaction flask containing magnetons, and the reaction was performed while stirring overnight at 75° C. The solvent was removed using the rotary evaporator and the product was separated by the thin layer chromatographic column (dichloromethane:methanol=10:1 by volume ratio) to obtain the following target products.

The intermediate product A was prepared using cis-1,4-cyclohexanediamine, the organic amine was didecylamine, the product was CN-2-10 (yield of 49%), and the structural characterization: 1H NMR (400 MHZ, CDCl3) δ 8.29 (s, 2H), 3.88 (s, 2H), 2.75 (s, 4H), 2.53 (s, 8H), 2.40 (s, 4H), 1.73 (s, 4H), 1.48 (s, 4H), 1.25 (s, 64H), 0.87 (t, J=6.7 Hz, 12H).

The intermediate product A was prepared using cis-1,4-cyclohexanediamine, the organic amine was dodecylamine, the product was CN-2-12 (yield of 35%), and the structural characterization: 1H NMR (400 MHZ, CDCl3) δ 8.12 (s, 2H), 3.88 (s, 2H), 2.84 (s, 4H), 2.59 (s, 8H), 2.48 (s, 4H), 1.72 (s, 4H), 1.52 (s, 4H), 1.25 (s, 80H), 0.87 (s, 12H).

### Example 3: Preparation of mRNA-LNPs

The ionizable lipids, cholesterol, PEG, helper lipid were dissolved in ethanol, and mRNA encoding an expression of EGFP (EGFP IVT mRNA purchased from VectorBuilder, m1Ψ modified) was dissolved in a citrate buffer at a pH=4.0. The mixed lipid solution and the mRNA solution were mixed according to the prescription in Table 1 to obtain mRNA-LNPs.

In Table 1, the concentration of the mRNA was 1 μg/μL, the volume was 4 μL and the total volume of an aqueous phase was 90 μL; the concentration of the ionizable lipid was 10 μg/μL, the volume was 4 μL and total volume of an organic phase was 45 μL; and the concentration of the sterol was 5 μg/μL, the concentration of the PEG was 5 μg/μL, the concentration of the helper lipid was 5 μg/μL; and the weight ratio of the ionizable lipid to the mRNA was about 10:1. The mixing method was a microfluidic method.

TABLE 1

| | Ionizable | | | Helper | Sterol | PEG | DOPE | Ethanol |
|---|---|---|---|---|---|---|---|---|
| LNP | lipid | Sterol | PEG | lipid | (μL) | (μL) | (μL) | (μL) |
| 1 | TN-2-10 | Cholesterol | DMG-PEG | DOPE | 5.05 | 0.82 | 4.85 | 15.28 |
| 2 | TN-2-10 | Cholesterol | DMG-PEG | DOPE | 6.31 | 1.64 | 9.71 | 8.35 |
| 3 | TN-2-10 | Cholesterol | DMG-PEG | DOPE | 3.03 | 0.49 | 2.91 | 19.57 |
| 4 | TN-2-10 | Cholesterol | DMG-PEG | DOPE | 3.78 | 0.98 | 5.83 | 15.41 |
| 5 | TN-2-10 | Cholesterol | DMG-PEG | DOPE | 4.54 | 2.46 | 8.74 | 10.27 |
| 6 | TN-2-10 | Cholesterol | DMG-PEG | DOPE | 5.30 | 4.91 | 11.65 | 4.14 |
| 7 | TN-2-10 | Cholesterol | DMG-PEG | DOPE | 2.16 | 0.35 | 2.08 | 21.41 |
| 8 | TN-2-10 | Cholesterol | DMG-PEG | DOPE | 3.24 | 1.75 | 6.24 | 14.76 |
| 9 | TN-2-10 | Cholesterol | DMG-PEG | DOPE | 3.78 | 3.51 | 8.32 | 10.39 |
| 10 | TN-2-10 | Cholesterol | DMG-PEG | DOPE | 2.52 | 1.36 | 4.85 | 17.26 |
| 11 | TN-2-10 | Cholesterol | DMG-PEG | DOPE | 2.94 | 2.73 | 6.47 | 13.86 |
| 12 | TN-2-18-12 | Cholesterol | DMG-PEG | DOPE | 3.77 | 0.61 | 3.63 | 17.99 |
| 13 | TN-2-18-12 | Cholesterol | DMG-PEG | DOPE | 4.71 | 1.22 | 7.25 | 12.81 |
| 14 | TN-2-18-12 | Cholesterol | DMG-PEG | DOPE | 5.66 | 3.06 | 10.88 | 6.40 |
| 15 | TN-2-18-12 | Cholesterol | DMG-PEG | DOPE | 2.26 | 0.37 | 2.18 | 21.19 |
| 16 | TN-2-18-12 | Cholesterol | DMG-PEG | DOPE | 2.83 | 0.73 | 4.35 | 18.09 |
| 17 | TN-2-18-12 | Cholesterol | DMG-PEG | DOPE | 3.39 | 1.83 | 6.53 | 14.24 |
| 18 | TN-2-18-12 | Cholesterol | DMG-PEG | DOPE | 3.96 | 3.67 | 8.71 | 9.67 |
| 19 | TN-2-18-12 | Cholesterol | DMG-PEG | DOPE | 1.62 | 0.26 | 1.55 | 22.57 |
| 20 | TN-2-18-12 | Cholesterol | DMG-PEG | DOPE | 2.83 | 2.62 | 6.22 | 14.33 |
| 21 | TN-2-18-12 | Cholesterol | DMG-PEG | DOPE | 1.89 | 1.02 | 3.63 | 19.47 |
| 22 | TN-2-18-12 | Cholesterol | DMG-PEG | DOPE | 2.20 | 2.04 | 4.84 | 16.93 |
| 23 | CN-2-10 | Cholesterol | DMG-PEG | DOPE | 5.05 | 0.82 | 4.85 | 15.28 |
| 24 | CN-2-10 | Cholesterol | DMG-PEG | DOPE | 6.31 | 1.64 | 9.71 | 8.35 |
| 25 | CN-2-10 | Cholesterol | DMG-PEG | DOPE | 3.03 | 0.49 | 2.91 | 19.57 |
| 26 | CN-2-10 | Cholesterol | DMG-PEG | DOPE | 3.78 | 0.98 | 5.83 | 15.41 |
| 27 | CN-2-10 | Cholesterol | DMG-PEG | DOPE | 4.54 | 2.46 | 8.74 | 10.27 |
| 28 | CN-2-10 | Cholesterol | DMG-PEG | DOPE | 5.30 | 4.91 | 11.65 | 4.14 |
| 29 | CN-2-10 | Cholesterol | DMG-PEG | DOPE | 2.16 | 0.35 | 2.08 | 21.41 |
| 30 | CN-2-10 | Cholesterol | DMG-PEG | DOPE | 3.24 | 1.75 | 6.24 | 14.76 |
| 31 | CN-2-10 | Cholesterol | DMG-PEG | DOPE | 3.78 | 3.51 | 8.32 | 10.39 |
| 32 | CN-2-10 | Cholesterol | DMG-PEG | DOPE | 2.52 | 1.36 | 4.85 | 17.26 |
| 33 | CN-2-10 | Cholesterol | DMG-PEG | DOPE | 2.94 | 2.73 | 6.47 | 13.86 |

### Example 4: Particle Size and Potential Characterization of mRNA-LNPs

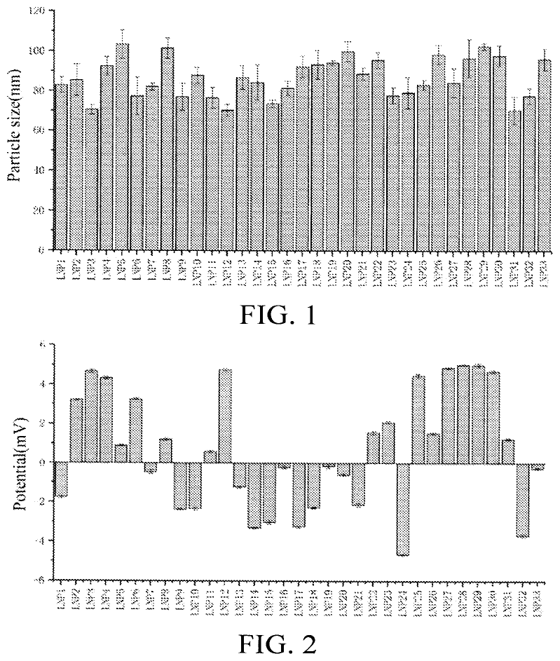

The particle sizes and potentials of the LNPs were detected using a dynamic light scattering in a 90° backscatter detection mode using Malvern Zetasizer Nano ZS. The results were shown in FIGS. 1 and 2. The particle size range of the LNPs shown in FIG. 1 was 70-110 nm, mostly 80-100 nm, which was consistent with a nano-drug scale, demonstrating that the prepared LNPs can smoothly pass through an intercellular space and had an excellent nucleic acid drug delivery performance. FIG. 2 showed that the (potential was between −6 mV to 6 mV, proving that an LNP preparation system was stable and not easy to aggregate or settle.

### Example 5: Encapsulation Efficiency Characterization of mRNA-LNPs

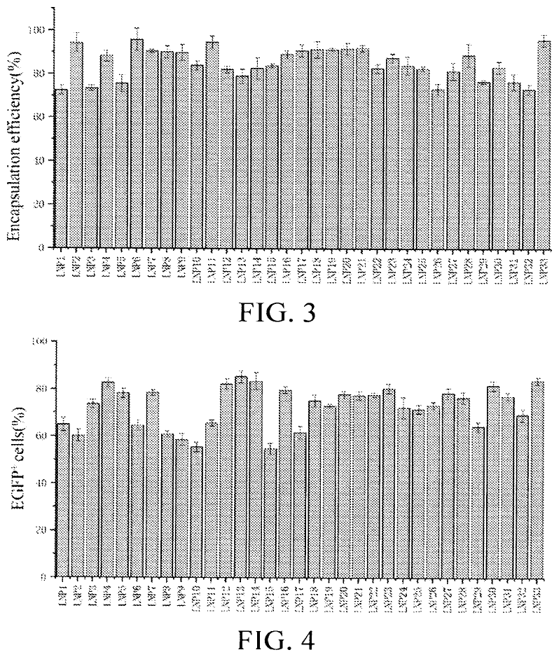

The encapsulation efficiency of the LNPs was determined using a Quant-iT™ RiboGreen® RNA reagent and a multi-mode microplate detection system Mutimode Plate Reader (EnSight). The Quant-iT™ RiboGreen® RNA reagent was not permeable to the LNPs, and therefore only free nucleic acids which were not entrapped by the LNPs can be bound. Triton-100 is a surfactant commonly used as a demulsifier, and the LNPs obtained by treating with 2% Triton-100 can release the entrapped nucleic acids to obtain the total nucleic acid amount. The drug loading amount was obtained by calculating the difference of the nucleic acid amount before and after the demulsification, and then the drug loading amount was divided by the total nucleic acid amount to obtain the encapsulation efficiency. The encapsulation efficiency of the series of the products was measured to be between 60% and 95%, and was shown in FIG. 3.

### Example 6: Transfection Efficiency Characterization of mRNA-LNPs

Hep3B cells in the logarithmic growth phase were inoculated to a 6-well cell plate (200,000 cells/well) for culture overnight, when the cell density reached 80% or more, the culture medium was discarded and washed 3 times using 1×PBS, 1 mL of EGFP mRNA-LNPs (1 μg/mL) solution prepared by a serum-free DMEM culture medium was added into cell wells, and 3 duplicate wells were arranged. After 6 h, the culture medium was discarded and replaced by a normal DMEM medium with serum, the culture was continued for 24 h, and the fluorescence ratio of the cells was detected using a flow cytometer. The result was shown in FIG. 4 that the transfection efficiency was between 10% and 90%. The TN series had higher transfection efficiency and good transfection performance.

The above description is merely a preferred embodiment of the present invention and is not intended to limit the present invention, and various changes and modifications may be made by those skilled in the art. Any modifications, equivalent substitutions, improvements and the like made within the spirit and principle of the present invention should be included within the protection scope of the present invention.

1. An ionizable lipid based on cyclohexanediamine, being a compound represented by formula (a), a pharmaceutically acceptable salt of the compound represented by formula (a), a stereoisomer of the compound represented by formula (a), or a tautomer of the compound represented by formula (a), 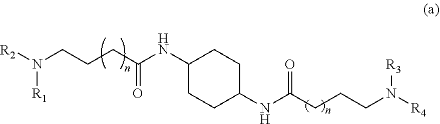
wherein n is 0; and
R₁, R₂, R₃ and R₄ are independently selected from C₈-C₂₄ alkyl and C₈-C₂₄ alkenyl.

2. The ionizable lipid based on cyclohexanediamine according to claim 1, comprising the following compounds:

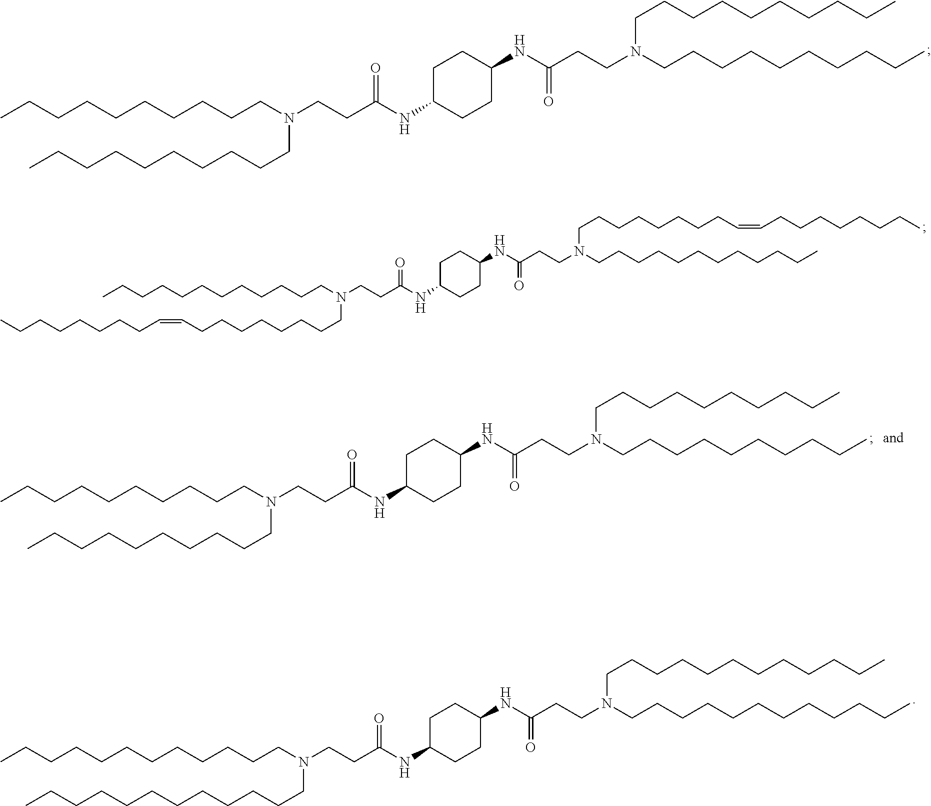

3. A method for preparing the ionizable lipid based on cyclohexanediamine according to claim 1, comprising the steps of obtaining the compound represented by formula (a) according to the following reaction formula, 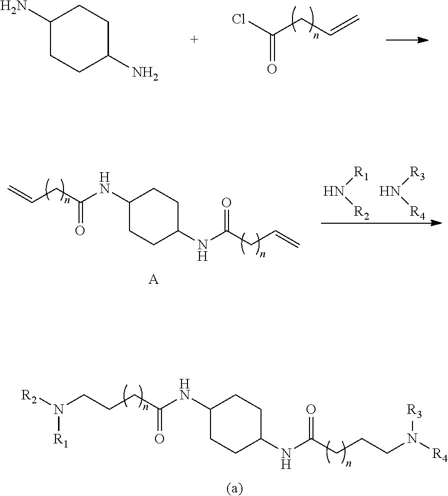
wherein n is 0; and
R₁, R₂, R₃ and R₄ are independently selected from C₈-C₂₄ alkyl and C₈-C₂₄ alkenyl.

4. The method for preparing the ionizable lipid based on cyclohexanediamine according to claim 3, wherein in the process of preparing an intermediate product A, materials are added under the ice bath condition and then reacted at room temperature; or
in the process of preparing the compound represented by formula (a) using the intermediate product A, the reaction temperature is 70-85° C.

5. A lipid nanoparticle, comprising the ionizable lipid based on cyclohexanediamine according to claim 1, a helper lipid, a sterol, and a PEG lipid.

6. The lipid nanoparticle according to claim 5, wherein the mole percent of the ionizable lipid based on cyclohexanediamine is 30-50%.

7. A pharmaceutical composition, comprising the ionizable lipid based on cyclohexanediamine according to claim 1 or the lipid nanoparticle comprising the ionizable lipid based on cyclohexanediamine, and an active ingredient, wherein the active ingredient is a nucleic acid drug.
